(19) United States
(12) Patent Application Publication
(10) Pub. No.: US 2019/0224124 A1
(43) Pub. Date: Jul. 25, 2019

(54) Methods and Kits for Reducing the Susceptibility of Lipoprotein Particles to Atherogenic Aggregation Induced by Arterial-Wall Enzymes

(71) Applicant: Kevin Jon Williams, Wynnewood, PA (US)

(72) Inventor: Kevin Jon Williams, Wynnewood, PA (US)

(21) Appl. No.: 16/328,783

(22) PCT Filed: Aug. 30, 2017
(86) PCT No.: PCT/US17/49351

§ 371 Date: Feb. 27, 2019

Related U.S. Application Data
(60) Provisional application No. 62/382,368, filed on Sep. 1, 2016.

(51) Int. Cl. A61K 9/127; A61K 9/00; A61K 38/17; A61P 9/00

(52) U.S. Cl. CPC A61K 9/127; A61P 9/00; A61K 38/1709; A61K 9/0029

(57) ABSTRACT

Methods of decreasing, in a human or other animal, the susceptibility of atherogenic particles to aggregation induced by sphingomyelinase, the methods comprising administering vesicles to the animal. Also similar methods directed at the formation of cholesterol crystals, abnormal cholesterol enrichment of cell membranes, and denaturation of Apo B.

### CROSS REFERENCE TO RELATED APPLICATIONS

This application claims the benefit of U.S. provisional application Ser. No. 62/382,368, filed Sep. 1, 2016. The entire disclosure of the foregoing provisional application is incorporated herein by reference in its entirety.

### FIELD OF THE INVENTION

The field of the invention is reducing, in humans, the susceptibility of low-density lipoprotein particles (LDL) and similar particles to aggregation induced by arterial-wall enzymes, such as a sphingomyelinase.

### BACKGROUND OF THE INVENTION

The ability to reduce the extent of atherosclerotic lesions in humans is a major goal of modern medicine.

Despite the successes of plasma LDL-lowering therapies in the treatment of atherosclerosclerotic cardiovascular disease (ASCVD), patients treated with optimal statin therapy (1, 2) and even the new PCSK9 inhibitors (3, 4) exhibit considerable residual risk for ASCVD events. There is a need for new approaches beyond the current tools for lowering plasma LDL levels.

Low-density lipoprotein particles (LDL) and related atherogenic lipoproteins normally enter and leave the arterial wall. Atherosclerosis arises from the retention, or trapping, of some fraction of these lipoproteins within the arterial wall, chiefly by their binding to molecules of the extracellular matrix, especially proteoglycans, in the arterial intima (5). The retained atherogenic lipoproteins become modified, and a key modification is aggregation (a process that can also include particle fusion) (6). Once these lipoproteins, each of which contains one molecule of apolipoprotein B (apoB), become aggregated, their egress from the arterial wall becomes unlikely. Their movement is sterically hindered by their larger size, and their affinity for arterial matrix increases. Moreover, aggregated apoB-lipoproteins are avidly taken up by local macrophages, loading them with cholesterol, thereby producing “foam cells,” a hallmark of atherosclerosis. Thus, aggregation of LDL and related lipoproteins within the arterial wall is a key step in the development and progression of atherosclerosis.

This aggregation is likely mediated by sphingomyelinase (SMase): a deficiency of secretory SMase (a product of the acid sphingomyelinase gene) has been linked to a reduction in LDL retention and atherosclerotic lesions in hypercholesterolemic mice, through effects within the arterial wall, without changing plasma concentrations of apoB-lipoproteins (7). Other arterial-wall enzymes may also contribute, such as other phospholipases (such as a phospholipase A₂) and lipoprotein lipase (the latter acting primarily as physical bridge). There is a need for a method to alter LDL and related lipoproteins in vivo, to make these particles far less susceptible to aggregation and therefore less atherogenic.

Dispersion of phospholipids (“PLs”) such as lecithins (phosphatidylcholines) into aqueous media has been shown to result in the self-assembly of the PLs into multilamellar liposomes, i.e., vesicles comprised of concentric spherical bilayers (also known as multilamellar vesicles or MLVs). A variety of manufacturing methods, such as extrusion (e.g., the LIPEX® Extruder) and high-shear and/or high-pressure methods (e.g., Microfluidizer® homogenization technology), are available to produce unilamellar vesicles (meaning vesicles comprised of a single lipid bilayer) of defined sizes. Unilamellar vesicles of at least 50 nm diameter are referred to here as large ‘empty’ vesicles (LEVs), according to common nomenclature, because they do not need to contain an encapsulated drug for the uses herein (nevertheless, encapsulated drugs are also contemplated). LEVs have sometimes also been referred to as large unilamellar vesicles (LUVs), in contrast to small unilamellar vesicles (SUVs), which are typically around 30 nm in diameter. After parenteral administration, typically intravenously, to experimental animals or human subjects, even cholesterol-free or cholesterol-poor LEVs at sufficient doses remain as intact particles in the circulation and have the ability to extract cholesterol from peripheral tissues (See discussion in (8)).

An effect of LEVs on the susceptibility of LDL and related atherogenic particles (those that have apolipoprotein B as their primary apolipoprotein) to SMase-induced aggregation, however, has not been previously shown. Such a demonstration would be useful as it would create additional methods for therapeutic interventions against the development and progression of atherosclerotic lesions. The present invention is based on such demonstration, one that measured the susceptibility of plasma LDL to SMase-induced aggregation in vitro. It will be seen that, in LDL from LEV-treated animals, this susceptibility has been greatly reduced.

The primary apolipoprotein of LDL is apoB. Other atherogenic lipoprotein particles that contain apoB are also susceptible to SMase. As a result, the beneficial effect of LEVs on the susceptibility of LDL to aggregate when exposed to SMase can be expected to occur also with those other atherogenic particles that contain apoB (collectively, LDL and these other atherogenic particles are sometimes referred to as ‘apoB-containing lipoproteins’ or more simply ‘apoB-lipoproteins’). Those other atherogenic particles are remnant lipoproteins, cholesterol- and triglyceride-rich remnant lipoproteins (together, referred to as C-TRLs), very low-density lipoprotein (VLDL), small VLDL (sVLDL), cholesterol-rich remnant lipoproteins, ß-VLDL, VLDL remnants, chylomicron remnants, postprandial remnants, intermediate-density lipoprotein (IDL), lipoprotein(a) [Lp(a)], and triglyceride-rich remnant lipoproteins (TRLs). Although chylomicrons also contain apoB, chylomicrons are generally too large to start with to efficiently enter the arterial wall and cause atherosclerosis (see Borén J and Williams K J. The central role of arterial retention of cholesterol-rich apoB-containing lipoproteins in the pathogenesis of atherosclerosis: a triumph of simplicity Curr Opin Lipidol. 2016, in press. doi: 10.1097/MOL.0000000000000330).

LEVs have advantages compared to MLVs and SUVs. By having only a single phospholipid bilayer, most of the lipid content of LEVs is directly exposed, i.e., available to beneficially alter LDL and other apoB-lipoproteins. By contrast, the multilamellar structure of MLVs means that internal bilayers are shielded and therefore less efficient at altering LDL and other atherogenic lipoproteins to become less susceptible to aggregation.

Furthermore, SUVs have a harmful side-effect of suppressing LDL receptor expression in the liver, thereby increasing plasma concentrations of LDL (8). LEVs avoid the side-effect of suppressing LDL receptors and hence the side-effect of raising plasma concentrations of LDL (8).

The present inventions are also relevant to the formation of crystals of unesterified cholesterol (“cholesterol crystals”) and other harmful materials within the arterial wall. Such other harmful materials include, but are not limited to dangerous lipids and lipid-rich structures, modified apoB₁₀₀ and apoB₄₈ and their fragments.

Excess unesterified cholesterol from retained and aggregated apoB-lipoproteins, such as LDL, remnant lipoproteins, Lp(a), and small VLDL, within the arterial wall has been shown to cause or accelerate a number of maladaptive responses. These maladaptive responses include, but are not limited to, the reported formation of unesterified cholesterol crystals and microcrystals that then activate the inflammasome, particularly the NLRP3 inflammasome, that then causes activation and release of interleukin (IL)-1 beta (IL1ß), IL6, with resulting downstream harm (references²¹⁻²⁵). Typical of disclosures in the art is Guarino et al. who reported a combined effect of sphingomyelinase and cholesterol esterase in promoting cholesterol crystal nucleation from enzymatically modified LDL.²¹ Also, Haka et al. reported that macrophages, a prominent cell type in the atherosclerotic plaque, latch onto extracellular aggregated LDL and digest regions of the aggregated LDL that are adjacent or nearby to the macrophages, resulting in the release of significant amounts of unesterified cholesterol.²⁶ This unesterified cholesterol is a likely source for the formation of extracellular cholesterol crystals.²⁷

Additional maladaptive responses to cholesterol from retained and aggregated apoB-lipoproteins include abnormal unesterified cholesterol-enrichment of cell membranes²⁶ that then activates phagocytic pathways,²⁸ toll-like receptors,²⁹ the inflammasome³⁰ and enzymes that produce pro-retentive arterial matrix.³¹⁻³³

In addition, delipidated, or denatured, apoB activates proatherogenic T-cell hybridomas (as indicated by release of IL2, [³H]thymidine incorporation, and other known methods).³⁴ SMase-induced aggregation of LDL causes substantial apoB denaturation.³⁵

It has not, however, been disclosed in the art that LEVs can be employed to inhibit the aggregation of apoB-lipoproteins and therefore counter the formation of cholesterol crystals, abnormal cholesterol-enrichment of cell membranes, denaturation of apoB, and the development of other harmful materials derived from apoB-lipoproteins aggregated in the presence of sphingomyelinase.

The present invention addresses the need for methods and compositions to target the initial steps in provoking these maladaptive immune responses.

Furthermore, the present invention avoids side-effects, including immune suppression and other immune derangements, that arise from current methods to inhibit IL1ß, IL6, and other immune mediators or functions. ^{24, 25, 36, 37} For example, a recent clinical trial showed that an inhibitor of IL1ß administered to cardiovascular patients was associated with a higher incidence of fatal infection in these patients than was placebo. ^{25, 36, 37} Moreover, current methods directed towards suppressing immune functions fail to address the root cause of apoB-lipoprotein aggregation and retention, and the formation of cholesterol crystals, abnormally cholesterol-enriched membranes, denatured apoB, and other harmful lipoprotein-derived material. Instead the present invention is disease-specific, i.e., directed to processes that occur in the initiation, progression, and destabilization of atherosclerotic plaques. As a result, the present invention represents a major advance in addressing the clinical problem of residual or unrecognized cardiovascular risk.

### BRIEF DESCRIPTION OF THE DRAWINGS

FIG. 1 is a graph showing the relationship of mean aggregate size in nanometers of LDL particles (vertical axis) to the number of hours (horizontal axis) that purified LDL preparations were incubated with SMase and allowed to aggregate. (Nearly all of the enzymatic digestion of sphingomyelin is expected to occur early on; the result is a change in the conformation of apoB that leads to gradual aggregation during the next 18-24 hours—see references (9) and (10).)

FIG. 2 shows the data from 0 to 5 hours from FIG. 1, using expanded horizontal- and vertical-axis scales.

FIG. 3 shows the mean diameter and narrow size distribution, as assessed by dynamic light scattering, of a typical preparation of POPC LEVs made by extrusion. The horizontal axis shows the diameter of the particles in nanometers (nm). The count rate was 235.2 kilo counts per second (kcps). The “Z-average diameter size” was 110.8 nm. The polydispersity index (“PDI”) was 0.041, and the PDI width was 22.38 nm. The mean diameter of the peak area was 116.9 nm.

FIG. 4 shows the decrease in the molar ratio of sphingomyelin to phosphatidylcholine (SM:PC) in the LDL of LEV-treated mice compared with LDL of saline-treated mice.

### BRIEF SUMMARY OF THE INVENTION

The invention is the administration of LEVs to humans (and other animals) to decrease the susceptibility of LDL and related lipoproteins to form aggregates. Aggregates of these lipoproteins are key contributors to intra-arterial accumulation of cholesterol and other harmful material and hence the formation of atherosclerotic plaques that cause heart attacks, strokes, peripheral vascular disease, and other forms of atherosclerotic cardiovascular disease (ASCVD).

In a variation of the foregoing, the LEVs are administered in order to inhibit the formation of crystals of unesterified cholesterol, abnormal cholesterol-enrichment of cell membranes, denaturation of apoB, the development of other harmful materials derived from apoB-lipoproteins aggregated in the presence of sphingomyelinase, inflammasome activation (particularly the NLRP3 inflammasome), activation of proatherogenic T-cells, release of harmful cytokines (such as IL13 and IL6), plaque progression and destabilization, and release of C-reactive protein (“CRP”). These effects are achieved without immune suppression.

### DETAILED DESCRIPTION

### Terminology

A human is considered herein to be an “animal.”

“ApoB” refers to apolipoprotein-B, a term that comprises both the full-length form, apoB₁₀₀, and the truncated form, apoB₄₈.

“LDL” and “LDL particles” both refer to low-density lipoprotein particles.

“SMase” refers to sphingomylenase. As used herein it is a general abbreviation for all sphingomyelinases. The main sphingomyelinase in the arterial wall involved in atherosclerotic plaque development is the secretory SMase (“S-SMase”).

“VLDL” refers to very-low-density lipoprotein particles.

“IDL” refers to intermediate-density lipoprotein particles.

“Lp(a)” refers to lipoprotein(a), a form of LDL that includes the apolipoprotein(a).

“C-TRL” refers collectively to cholesterol- and triglyceride-rich apoB-containing lipoproteins, a group that comprises, in particular, cholesterol- and triglyceride-rich apoB-containing remnant lipoproteins.

“TRL” refers to triglyceride-rich lipoproteins, a group that comprises triglyceride-rich apoB-containing remnant lipoproteins.

“β-VLDL” (i.e., beta-VLDL), refers specifically to a type of cholesterol-rich remnant lipoprotein particle seen in type III dyslipoproteinemia and in apoE knock-out mice.

“sVLDL,” “small VLDL”, and “sVLDL particles” refers to small very-low-density lipoprotein particles.

“LEV” stands for “large empty vesicle.” LEVs have also been referred to as “LUV,” which stands for large unilamellar vesicle. The terms “LUV” and “LEV” are used interchangeably.

“POPC” stands for palmitoyloleoylphosphatidylcholine a/k/a 1-palmitoyl, 2-oleyl phosphatidylcholine, a/k/a palmitoyl-oleoyl phosphatidyl choline.

The terms “vesicle” and “liposome” are used interchangeably in this document.

The term “atherogenic lipoprotein particle” as used herein refers to atherogenic apolipoprotein particles that comprise apolipoprotein B.

“TG-rich apoB-lipoproteins” as used herein refers to atherogenic TG-rich apoB-lipoproteins.

### Aspects of the Invention

In a general aspect, the invention is a method (“the method of the invention”) of decreasing the susceptibility of atherogenic lipoprotein particles to aggregation induced by a sphingomyelinase (SMase) in an animal that comprises a SMase and said atherogenic lipoprotein particles, said method comprising administering vesicles (or liposomes) to said animal so as to cause a decrease in said susceptibility, provided said vesicles or liposomes do not comprise significant amounts of sphingomyelin or unesterified cholesterol, and wherein a human is considered to be an animal, and wherein the animal comprises a closed circulatory system that comprises an artery.

“Do not comprise significant amounts”, as related to sphingomyelin will, on the average, mean that the vesicles or liposomes comprise sphingomyelin to a level such that the sphingomyelin:phospholipid (SM:PL) molar ratio in the LEVs is below 0.07. Preferably that SM:PL molar ratio is not more than 0.033, more preferably not more than 0.0165, even more preferably not more than 0.0033, and most preferably not more than 0.00165.

“Do not comprise significant amounts” as related to unesterified cholesterol will, on the average, means that the vesicles or liposomes comprise unesterified cholesterol to a level such that that the unesterified cholesterol: phospholipid (UC:PL) molar ratio in the LEVs is below 0.1. Preferably that UC:PL molar ratio is not more than 0.05, more preferably not more than 0.01, even more preferably not more than 0.003, and most preferably not more than 0.001.

In a subset of the foregoing aspects of the method of the invention, the method is not applied to a human with dyslipidemia. Of interest would be a person with ASVCD who is receiving therapy with a statin, ezetimibe, and/or a PCSK9 inhibitor and has achieved therapeutic targets for LDL or apoB concentrations in plasma. That person may no longer have a dyslipidemia, yet still has atherosclerotic plaques and likely still has residual cardiovascular risk. Therefore, of particular interest is an individual at high risk (recognized or unrecognized) of an ASCVD event but who at the moment no longer has a dyslipidemia, owing to successful LDL-lowering therapies.

In an aspect of particular interest, the method of the invention is applied to a human.

In an aspect of additional interest, the method of the invention is applied to a human at (moderate, high, or very high) atherosclerotic cardiovascular risk. Such a human can be identified by the presence of one or more characteristics selected from the group consisting of known presence of atherosclerotic cardiovascular disease (ASCVD; for example as indicated by a ASCVD risk calculator), high plasma concentrations of LDL, high plasma concentrations of apoB, high plasma concentrations of an apoB-lipoprotein, high blood pressure, history of high blood pressure, smoking, history of smoking, diabetes mellitus, the metabolic syndrome, components of the metabolic syndrome, the atherometabolic syndrome, a high plasma concentration of C-reactive protein, a high coronary artery calcium score, an abnormal carotid ultrasound, an imaging method indicating vulnerable plaque, an imaging method showing macrophage activation in the arterial wall, an imaging method showing protease activity in the arterial wall, and an assay showing high susceptibility of LDL or other apoB-lipoproteins to aggregation and/or arterial retention. Such humans can be identified by the presence of an orphan or a common disease that predisposes one to accelerated ACSVD.

In a subset of the foregoing aspect of additional interest, the method is not applied to a human with dyslipidemia.

The aforementioned “orphan or common disease” that predisposes a human to ACSVD can be selected from the group consisting of familial hypercholesterolemia, heterozygous familial hypercholesterolemia, homozygous familial hypercholesterolemia, ‘polygenic’ familial hypercholesterolemia, type IIa hyperlipidemia, type IIb hyperlipidemia, type III hyperlipidemia, type IV hyperlipidemia, a disease caused by a recessive, co-dominant, or dominant mutation that causes hypercholesterolemia, combined hyperlipidemia, familial combined hyperlipidemia (FCHL), a condition with high plasma concentrations of Lp(a), and a condition with high plasma concentrations of apoB. Also contemplated is a condition associated with increased susceptibility of plasma LDL and/or other apoB-lipoproteins to aggregation upon exposure to SMase.

A subset of those orphan or common diseases are familial hypercholesterolemia, heterozygous familial hypercholesterolemia, homozygous familial hypercholesterolemia, ‘polygenic’ familial hypercholesterolemia, type IIa hyperlipidemia, type IIb hyperlipidemia, type III hyperlipidemia, type IV hyperlipidemia, a disease caused by a recessive, co-dominant, or dominant mutation that causes hypercholesterolemia, combined hyperlipidemia, and familial combined hyperlipidemia (FCHL).

Another subset of such orphan and common diseases are ones that predispose the human to accelerated ACSVD disease such as a condition associated with high plasma (“higher than normal”) concentrations of apoB. Plasma concentrations of apoB considered to be higher than desirable or recommended depend on cardiovascular risk; it is generally known in the art that apoB levels at <80 and <100 mg/dL can be reasonable goals for subjects with very high and high CV risk, respectively (11 at 2352). For purposes of this patent application an apoB level of 100 mg/dL or higher is considered higher than normal.

Another subset of such orphan and common diseases are ones that predispose the human to accelerated ACSVD disease such as a condition associated with higher than normal susceptibility of plasma LDL and/or other apoB-lipoproteins to aggregation upon exposure to SMase.

ACSVD risk estimations are well-known in the art and have been recently summarized in the literature (11).

Preferably, the vesicles are administered parenterally. Preferably, the vesicle is an LEV. Preferably, the vesicles comprise one or more phospholipids, provided the vesicles do not comprise significant amounts of sphingomyelin.

In particular embodiments of the method of the invention, the vesicles comprise a phospholipid that is selected from the group consisting of phosphatidylcholine (especially egg phosphatidylcholine), phosphatidylglycerol (especially egg phosphatidylglycerol), distearoylphosphatidylcholine, distearoylphosphatidylglycerol, and POPC.

In particular embodiments of the method of the invention, the atherogenic lipoprotein particle whose susceptibility to aggregation induced by SMase comprises apolipoprotein B. Those particles are preferably selected from the group consisting of LDL, remnant lipoproteins, cholesterol- and triglyceride-rich remnant lipoproteins (together, referred to C-TRLs), very low-density lipoprotein (VLDL), small VLDL (sVLDL), cholesterol-rich remnant lipoproteins, ß-VLDL, VLDL remnants, chylomicron remnants, postprandial remnants, intermediate-density lipoprotein (IDL), lipoprotein(a) [Lp(a)], and triglyceride-rich lipoproteins (TRL). It is understood that apolipoprotein B (apoB) refers to the full-length apopB₁₀₀ (secreted mostly from the liver in humans), as well as the truncated apoB₄₈ (secreted mostly from the intestine in humans).

LDL are particles of particular interest. It is understood that these lipoproteins can aggregate with their own kind and/or with other apoB-lipoproteins, e.g., LDL can aggregate with LDL, LDL particles can also make a mixed aggregate with C-TRLs, and so forth. Likewise, C-TRLs can aggregate with each other.

Preferably, in the method of the invention the total vesicle dose administered per kg of body weight of the human is in the range 10 mg/kg to 1600 mg/kg (preferably in the range 100 to 1600 mg/kg, most preferably in the range 300 mg/kg to 1000 mg/kg), said total dose either administered as a single dose or divided into multiple doses, wherein said multiple divided dosages are administered over at most a short time period (such as 24 hours); and wherein said total vesicle dose is administered at least once.

In some particular embodiments of the method and system of the invention, the susceptibility to aggregation of the atherogenic lipoprotein particles and/or their retention by arteries is determined using an assessment system, said assessment capable of measuring such susceptibility and/or retention. Such assessment systems are described in detail below in the section “Assessment system for measuring the susceptibility to aggregation of the atherogenic lipoprotein particles and/or their retention by arteries”.

In a related aspect, the invention is a method of measuring susceptibility of atherogenic lipoprotein particles to aggregation induced by a sphingomyelinase (SMase) in a human or other animal, said method comprising the steps of (1) obtaining a sample of plasma from a human or other animal to whom vesicles or liposomes have been administered; and (2) subjecting that sample to a test for susceptibility of its atherogenic lipoprotein particles to aggregation induced by a SMase; wherein said vesicles or liposomes do not comprise significant amounts of sphingomyelin or unesterified cholesterol.

In said method of measuring susceptibility of atherogenic lipoprotein particles to aggregation induced by a sphingomyelinase (SMase) in a human or other animal, the time between step (1) and the start of step (2) is preferably not more than 7 days, more preferably not more than 3 days, most preferably not more than one day. The plasma sample is preferably stored at not more than ambient temperature (e.g., about 25 degrees centrigrade (° C.) in the interval between step (1) and the start of step (2).

In a particular aspect of the method of the invention, the method is extended to comprise a step of modifying the vesicle (e.g., LEV) dose based on the results obtained using the assessment in the human or other animal such that if a dose (a reference dose) leads to a result selected from the group consisting of less aggregation, a change in atherogenic lipoprotein particle composition indicating less aggregation susceptibility, less retention in an arterial wall, an assessment of an adverse response in an artery to aggregated LDL or other apoB-lipoproteins, such as macrophage accumulation, activation, or M1 polarization, and/or expression of a protease, protease activity, tissue factor, or atherogenic cytokine, then the next LEV dose is decreased compared to the reference dose and/or the time interval between the reference dose and the next dose is increased compared to the time interval between the reference dose and the previous dose. Of course, if the reference dose was the first dose, then such a time interval adjustment would not be possible.

In regard to such a modification of the vesicle dose based on results from an assessment system, a dose is as discussed above—either a single dose or multiple doses administered over at most a short time period. Failure of an LEV dose to result in sufficiently less aggregation, less aggregation susceptibility, less retention, and/or an assessment of an adverse response in an artery to aggregated LDL or other apoB-lipoproteins, such as macrophage accumulation, activation, or M1 polarization, and/or expression of a protease, protease activity, tissue factor, or atherogenic cytokine indicates that an increase in dosage (higher amount and/or more frequent administration) should be considered. Moreover, it is highly desirable to initiate LEV treatment for a condition associated with increased susceptibility of plasma LDL and/or other apoB-lipoproteins to aggregation and/or whenever these assessments indicate increased susceptibility to aggregation of the atherogenic lipoprotein particles and/or their retention by an artery or arteries.

Consistent with the foregoing, an aspect of the invention is a method of modifying a vesicle dose in a human or other animal, said method comprising the steps of:

1) administering a dose (“reference dose”) of vesicles or liposomes to a human or other animal so as to change the susceptibility, in said human or other animal, of atherogenic particles to SMase-induced aggregation;

2) assessing a result in said human or other animal, based on a result obtained using an assessment system, said result selected from the group consisting of less aggregation, a change in atherogenic lipoprotein particle composition indicating less aggregation susceptibility, less retention in an arterial wall, an assessment of an adverse response in an artery to aggregated LDL or other apoB-lipoproteins, such as macrophage accumulation, activation, or M1 polarization, and/or expression of a protease, protease activity, tissue factor, or atherogenic cytokine; and

3) if the reference dose leads to a decrease in said susceptibility, then administering the next vesicle or liposome dose such that said next dose is smaller than the reference dose and/or the time interval between said reference dose and said next dose is less than the time interval between the reference dose and the dose preceding said reference dose. In a subset of the foregoing method, the method is not applied to a human with dyslipidemia.

In other aspects of the method of the invention, the liposomes or vesicles are administered with another medication. Such possible medications are discussed below.

In another aspect, the method of the invention is used to effect at least one change in the composition of the LDL (or other apoB-lipoprotein) of the human or other animal, said change selected from the group consisting of a decrease in the molar ratio of sphingomyelin to phosphatidylcholine (SM:PC), an increase in the molar fraction of PC that is POPC, a decrease in the ratio of unesterified cholesterol to phosphatidylcholine (UC:PC), a decrease in the lysoPC:PC ratio, an increase in the ratio of PC:protein, an increase in the ratio of POPC:protein, an increase in the ratio of PC to apoB, an increase in the ratio of POPC to apoB, an increase in the ratio of PC to cholesteryl ester (PC:ChE), an increase in the ratio of POPC:ChE, an increase in the ratio of PC to triglycerides (PC:TG), an increase in the ratio of POPC:TG, a decrease in the UC:protein ratio, and any other measures that indicate enrichment of LDL (or other apoB-lipoproteins) in PC and/or POPC, and/or depletion in SM, lysoPC, UC, and apoC-III.

In a related particular aspect of effecting at least one change in the composition of the LDL (or other apoB-lipoprotein), the vesicle or liposome used in the method comprises a phospholipid that is the same as the phospholipid whose molar fraction will be increased in the LDL or other apoB-lipoprotein.

In another general aspect, the invention is a kit (“the kit of the invention”). The kit is for decreasing the susceptibility of atherogenic lipoprotein particles to aggregation in a human (or other animal), said kit comprising:
(1) vesicles; and
(2) printed notice indicating that the kit can be used to decrease the susceptibility of atherogenic lipoprotein particles to aggregation in a human (or other animal),
   wherein the vesicles do not comprise significant amounts of sphingomyelin.

The kit is intended for decreasing the susceptibility of atherogenic lipoprotein particles to aggregation induced by SMase and that may be specified in the printed notice.

In particular aspects of the kit of the invention, the vesicle is an LEV. Preferably, the vesicles comprise one or more phospholipids, provided the vesicles do not comprise significant amounts of sphingomyelin or unesterified cholesterol. In particular aspects, the vesicles comprise a phospholipid that is selected from the group consisting of phosphatidylcholine (especially egg phosphatidylcholine), phosphatidylglycerol (especially egg phosphatidylglycerol), distearoylphosphatidylcholine, distearoylphosphatidylglycerol, POPC, combinations thereof, and derivatives thereof. POPC is a highly preferred phospholipid.

In particular aspects of the kit of the invention, the kit is intended to reduce the SMase-induced aggregation of atherogenic lipoprotein particles: LDL, remnant lipoproteins, cholesterol- and triglyceride-rich remnant lipoproteins (together, referred to C-TRLs), very low-density lipoprotein (VLDL), small VLDL (sVLDL), cholesterol-rich remnant lipoproteins, ß-VLDL, VLDL remnants, chylomicron remnants, postprandial remnants, intermediate-density lipoprotein (IDL), lipoprotein(a) [Lp(a)], and triglyceride-rich remnant lipoproteins (TRL).

In this context, the prefix “apo-” refers to a protein component of a lipoprotein, e.g., apolipoproteins can be isolated after the lipid of the lipoprotein has been removed.

The printed notice may be on sheet of paper, a label, or a package. The printed notice requirement of the kit of the invention is satisfied if the kit comprises a printed notice of where the user can go (for example to a website) to find out that the kit can be used to decrease the susceptibility of atherogenic lipoprotein particles to aggregation in a human (or other animal with a closed circulatory system).

In particular aspects, the kit of the invention is combined with an assessment system for measuring extent of aggregation of atherogenic lipoprotein particles and/or their retention in an artery, arteries, or arterial segment of a human or other animal. Such assessment systems are discussed above in relation to the methods of the invention, specifically as to systems that can be used to determine whether the LEV dose should be modified.

When the kit comprises an assessment system, it can be referred to as a system of the invention.

In a further aspect, the inventions are methods in which the vesicles (or liposomes) are administered in order to inhibit the formation of crystals of unesterified cholesterol, abnormal cholesterol-enrichment of cell membranes, denaturation of apoB, the development of other harmful materials derived from apoB-lipoproteins aggregated in the presence of sphingomyelinase, inflammasome activation (particularly the NLRP3 inflammasome), activation of proatherogenic T-cells, release of harmful cytokines (such as IL1β and IL6), plaque progression and destabilization, and release of C-reactive protein (“CRP”). In a related aspect, the inventions are methods of monitoring the efficacy of those methods. These effects are achieved without harmful immune suppression or other harmful immune derangements.

The aforementioned methods of monitoring efficacy include, but are not limited to, assays of apoB-lipoprotein accumulation within the arterial wall, apoB-lipoprotein aggregation within the arterial wall, cholesterol crystal formation within the arterial wall, inflammasome activation, inflammasome activation within the arterial wall, T cell activation, T cell activation within the arterial wall, release of harmful cytokines such as active IL1ß and IL6, release of IL2, and levels of the marker CRP.

An example of an assay for apoB-lipoprotein accumulation within the arterial wall is administration of labeled lipoproteins then assessment of the accumulation of their label within the arterial wall.

An example of an assay for apoB-lipoprotein aggregation within the arterial wall is administration of doubly labeled lipoproteins such that their aggregation either quenches or enhances the label.

An example of an assay for cholesterol crystal formation within the arterial wall is administration of labeled lipoproteins such that cholesterol nucleation enhances the signal (as in Guarino et al. 2004).²¹

An example of an assay for inflammasome activation is the release of related cytokines and downstream products, such as IL1ß, IL6, and CRP.

An example of an assay for inflammasome activation within the arterial wall is inflammasome-specific imaging.

An example of an assay for T-cell activation is the release of T-cell-specific cytokines.

An example of an assay for T-cell activation within the arterial wall is T-cell-specific imaging.

An example of an assay for release of harmful cytokines (such as IL1ß, IL6 and IL2) is a quantitative assay of their concentrations in plasma or serum or other body fluids.

An example of an assay for levels of CRP is well-known in the art and commercially available from routine clinical chemistry laboratories.

Assessment System for Measuring the Susceptibility to Aggregation of the Atherogenic Lipoprotein Particles and/or their Retention by Arteries

As noted above, in some particular embodiments of the method of the invention, the susceptibility to aggregation of the atherogenic lipoprotein particles and/or their retention by arteries is assessed in an assessment system.

Also as noted above, in some particular embodiments of the system of the invention, the susceptibility to aggregation of the atherogenic lipoprotein particles and/or their retention by arteries is assessed in an assessment system that forms part of the system of the invention.

For assessment of susceptibility to aggregation, such an assessment system is an assay in vitro and/or a system for assessment of those particles in vivo, and/or by an assay of the level of aggregation indicators in the human or other animal of interest.

The assay in vitro (or ex vivo) for assessment of susceptibility to aggregation is preferably selected from the group consisting of (1) measurement of the extent or rate of aggregation of LDL that has been isolated from plasma and then incubated ex vivo with an SMase (a/k/a the susceptibility of said LDL to aggregation induced by SMase), (2) the aggregation of apoB-lipoproteins isolated from plasma and then incubated ex vivo with an SMase, (3) the aggregation of LDL or another apoB-lipoprotein isolated from plasma and then incubated with an arterial-wall enzyme, (4) the aggregation of apoB-containing lipoproteins still in plasma isolated from the human or other animal or with other plasma components, (5) the aggregation of apoB-containing lipoproteins in the presence of plasma components to which one adds an enzyme such as an arterial wall enzyme, (6) the aggregation of apoB-lipoproteins by physical means (such as vortexing), (7) the aggregation of apoB-lipoproteins by oxidation, such as lipid peroxidation, (8) the aggregation of apoB-lipoproteins in the presence of a lipase and/or a protease (9) incubations of LDL or other atherogenic lipoproteins with arterial segments ex vivo and (10) a system for the determination of the composition of an apoB-lipoprotein.

A system for the determination of the composition of an apoB-lipoprotein would be any system that can determine the relative concentrations of the components of an apoB-lipoprotein. Susceptibility can be inferred from such a determination.

The arterial-wall enzyme used in the assay in vitro for assessment of susceptibility to aggregation is preferably selected from the group consisting of a SMase, a human SMase, a human recombinant SMase, a SMase used at an acidic pH, a phospholipase, a phospholipase A₂, a lipase, a cholesteryl esterase, a lysosomal acid lipase, a protease, a matrix metalloproteinase, a caspase, a furin, an intracellular protease, a calpain, the proteasome, a cathepsin, an extracellular protease, an intracellular hydrolase that is released from a cell.

It is understood that assays of LDL aggregability or composition (or of other atherogenic lipoproteins) can be automated, such as on a clinical autoanalyzer, automated mass spectrometry, nephelometry, ELISA and ELISA-like assays, turbidometric analyses, rate-zonal centrifugation, and/or dynamic light scattering (DLS).

The system for assessment of the retention of the atherogenic particles in the arteries in vivo is preferably selected from the group consisting of an assay of apoB-lipoprotein aggregation and/or retention within the arterial wall in vivo, an imaging method of retained and/or aggregated lipoproteins within the arterial wall, an assay of lipoprotein aggregation and/or retention in a healthy arterial segment, an assay of lipoprotein aggregation and/or retention in a diseased arterial segment, an imaging method (such as cardiac catheterization, intravascular ultrasound (IVUS), an MRI, an MRI with contrast, a CT scan, a scan with contrast, an imaging method with a contrast agent wherein said contrast agent comprises a nanoparticle, and a nuclear medicine study), a method that involves injection of said apoB-lipoprotein into an animal, a method that involves labeling of said apoB-lipoprotein followed by its injection into an animal (said animal comprising a human and a non-human animal), and a method that involves assessments of endogenous apoB-lipoproteins in vivo. Arterial retention of artificial nanoparticles is also contemplated (e.g., see Cormode D P, Frias J C, Ma Y, Chen W, Skajaa T, Briley-Saebo K, Barazza A, Williams K J, Mulder W J, Fayad Z A and Fisher E A. HDL as a contrast agent for medical imaging. Clin Lipidol. 2009; 4:493-500. doi: 10.2217/clp.09.38).

### Chemical Composition of LEVs

Any amphipathic material that allows a liposomal or micellar structure can be used to make the LEVs. Phospholipids are a preferred material. Inclusion of significant amounts (defined above) of sphingomyelin or unesterified cholesterol in the LEVs, however, is to be avoided, and that fact should be understood as a caveat in all discussions of LEV structure and composition herein.

Preferred phospholipids for use in formation of LEVs are phosphatidylcholine (especially egg phosphatidylcholine), phosphatidylglycerol (especially egg phosphatidylglycerol), distearoylphosphatidylcholine, distearoylphosphatidylglycerol, palmitoyl-oleoyl phosphatidyl choline (POPC), dimyristoylphosphatidylcholine, soybean phosphatidylcholine, soybean phosphatidylglycerol, lecithin, P,y-dipalmitoyl-a-lecithin, phosphatidylserine, phosphatidic acid, N(2,3di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, stearoyl-palmitoyl-phosphatidylcholine, di-palmitoyl-phosphatidylethanolamine, di-stearoyl phosphatidylethanolamine, di-myrstoyl-phosphatidylserine, di-oleyl-phosphatidylcholine, combinations thereof, and derivatives thereof. A person in the art would know that the list could be extended by using amphipathic compounds similar to those specified here.

Among the foregoing preferred phospholipids, highly preferred phospholipids for formation of LEVs are phosphatidylcholine (especially egg phosphatidylcholine), phosphatidylglycerol (especially egg phosphatidylglycerol), distearoylphosphatidylcholine, distearoylphosphatidylglycerol, POPC, combinations thereof, and derivatives thereof.

A very highly preferred phospholipid is POPC and, accordingly, LEVs that comprise POPC are very highly preferred.

It can be seen that phospholipid molecules that comprise phosphatidyl choline as part of their chemical composition are a preferred group.

In making the LEVs, the POPC or other phospholipid LEV component can be supplemented with small amounts of other lipids or molecules, such as sphingosine-1-phosphate (S1P) and/or specific classes of lysoPC that interfere with SMase-induced aggregation of LDL and related lipoprotein.

The liposomes may also be bound to a variety of proteins and polypeptides to increase the remodeling of endogenous LDL. Binding of apolipoproteins (apoproteins) to the liposomes is particularly useful. As used herein, “bound to liposomes” or “binding to liposomes” indicates that the subject compound is covalently or non-covalently bound to the surface of the liposome or contained, wholly or partially, in the interior of the liposome. Apoprotein A-I (apoA-I), apoprotein A-II (apoA-II), and apoprotein E (apoE) will generally be the most useful apoproteins to bind to the liposomes. These small, amphipathic, exchangeable apoproteins inhibit the aggregation of LDL and related atherogenic apoB-containing lipoproteins. ApoA-I mimetic peptides, such as the 4F peptide, are similarly of use. Other amphipathic peptides are of similar use in this invention.

Liposomes used in the methods, kits or systems of the present invention may be bound to molecules of apoprotein A-I, apoprotein A-II, lecithin-cholesterol acyltransferase, and/or small amphipathic peptides (such as apolipoprotein A-I mimetic peptides, the 4F peptide, and/or peptides that mimic amphipathic sequences from proteins or apoproteins, such as apoA-I, apoE, an apoC, apoJ, apoM, and apoB), singly or in any combination and molar ratio. Additional proteins or other non-protein molecules may also be useful to bind to the liposomes to enhance liposome stability, half-life, and other properties, as well as remodeling of LDL and related apoB-lipoproteins. These additional proteins or other non-protein molecules include, without limitation, polyethyleneglycol, alkylsulfates, ammonium bromide, and albumin. (The term, “without limitation”, means that there are other such proteins or other non-protein molecules beyond those listed.)

Non-phosphorus containing lipids may also be used in the liposomes of the compositions of the present invention. These include, without limitation, stearylamine, docecylamine, acetyl palmitate, and fatty acid amides. Additional lipids suitable for use in the LEVs of the present invention are well known to persons of skill in the art and are cited in a variety of well-known sources (see, for example, reference 12).

The LEV preparation can be supplemented for purposes of sterility and stability with compounds used with other drug preparations that are to be administered intravenously (iv).

However, synthetic, non-allergenic phospholipids are preferable to naturally occurring phospholipids. For example, synthetic POPC is preferable over egg PC.

### Examples of Previously Used LEVs

LEVs made from phosphatidyl choline have been successfully used in previous experiments related to cholesterol transport from peripheral tissue to the liver. (See discussion in reference (8)). Their production has been described in Rodrigueza et al. (8), where they were referred to as LUVs. There, an extrusion membrane with pores of about 100 nm (“nanometers”) in diameter created LUVs of about 123+/−35 nm. That distinguished them from small unilamellar vesicles “SUVs” that had a diameter of 34+/−30 nm.

Whereas the phosphatidyl choline referred to in reference 8 was isolated from eggs, synthetic phosphatidyl cholines can also be used if they are in the liquid (or liquid crystal) state (i.e., not in the gel or solid state) at body temperature (likely if they have at least one double bond in the fatty acyl side-chains) yet are resistant to oxidation (do not have many double bonds). An example is POPC. LEVs constructed from POPC were used in the examples below.

### Physical Chemical Properties of LEVs

Generally, it is desirable that the LEVs be composed of lipids that are liquid (or liquid-crystalline) at 37° C., often at 35° C., and even 32° C. Liposomes in the liquid-crystalline state typically accept and donate component molecules with LDL and related apoB-lipoproteins more efficiently than do liposomes in the gel (or solid) state. Because patients typically have a core temperature of about 37° C., liposomes composed of lipids that are liquid-crystalline at 37° C. are generally in a liquid crystalline state during treatment and, therefore, optimize remodeling of LDL and other harmful apoB-lipoproteins.

### Size of LEVs

It is preferred that the vesicle is an LEV. It is preferred the mean diameter of the LEVs, be at least 50 nm, more preferably at least 100 nm. Preferably the mean diameter of the LEVs is not more than 1000 nm (1.0 mm), more preferably not more than 250 nm, most preferably not more than 150 nm. Unilamellar vesicles are preferred over multilamellar vesicles, to facilitate exposure of liposomal components to LDL and related apoB-lipoproteins, to maximize remodeling of these particles.

Highly preferred sizes are ones that do not alter liver metabolism to raise total plasma LDL concentrations (8).

The size of the liposomal vesicles may be determined by dynamic light scattering (DLS), quasi-elastic light scattering (QELS) (13), size-exclusion chromatography, electron microscopy, and other methods well-known in the art. Average LEV diameter may, if desired, be reduced by sonication of formed LEVs and/or extrusion through membranes of smaller pore-sizes and/or high-shear technologies. Intermittent application of these methods may be alternated with DLS, QELS, or other assessments to optimize LEV formation. Methods exist to assess the lamellarity of phospholipid dispersions, such as ³¹P-nuclear magnetic resonance (NMR) to monitor the phospholipid phosphorous signal, before versus after addition of an impermeable paramagnetic or broadening reagent to the external medium, which will decrease the intensity of the initial ³¹P-NMR signal by an amount proportional to the fraction of lipid exposed to the external medium, which for large unilamellar liposomes, such as LEVs, should be essentially 50%. (14)

### Pharmaceutical Carriers

The LEV compositions of the present invention also comprise a pharmaceutically acceptable carrier. Many pharmaceutically acceptable carriers may be employed in the compositions of the present invention. Generally, normal saline will be employed as the pharmaceutically acceptable carrier, typically buffered, such as a phosphate-buffered saline. Other suitable carriers include, e.g., 0.4% saline, half-normal saline, 0.3% glycine, and the like, including glycoproteins for enhanced stability, such as albumin, apolipoproteins, globulin, etc. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride.

The concentration of liposomes in the carrier may vary. Generally, the concentration will be about 20-500 mg of liposomal lipid per ml of carrier, usually about 50-200 mg/ml, and most usually about 100-200 mg/ml. Persons of skill may vary these concentrations to optimize treatment with different liposomal components or of particular patients. For example, the concentration may be increased to lower the fluid load associated with treatment. This may be particularly desirable in patients having atherosclerosis-associated congestive heart failure or severe hypertension. Alternatively, liposomes composed of irritating lipids may be diluted to low concentrations to lessen inflammation at the site of administration.

### Administration of LEVs

Typically, the liposomes will be administered via a peripheral vein for convenience. Sometimes, the LEVs will be administered into a large central vein, such as the superior vena cava or inferior vena cava, to allow highly concentrated solutions to be administered into large volume and flow vessels. Additionally, the LEVs may also be administered via a variety of other routes that allow them access to plasma apoB-containing lipoproteins or to intra-arterial apoB-containing lipoproteins. In this sense, “access” can mean direct access or indirect access.

The mode of LEV administration is preferably selected from the group consisting of parenteral administration, intravenous administration, intra-arterial administration, intramuscular administration, subcutaneous administration, transdermal administration, intraperitoneal administration, intrathecal administration, via lymphatics, intravascular administration—including administration into capillaries, arteriovenous shunts, and vascular stents for long-duration release, rectal administration, administration via a chronically indwelling catheter, and administration via an acutely placed catheter.

The frequency of administration and the dose administered on each occasion will be chosen so as to use the minimum dose needed to achieve the maximum beneficial effect on a person without significant side effects. This choice will be facilitated by the use of an assessment system for measuring extent of aggregation of atherogenic lipoprotein particles and/or their susceptibility to aggregation and/or their retention in the arteries of a human or other animal. Such assessment systems are discussed above in relation to the methods of the invention, specifically as to systems that can be used to determine whether the LEV dose should be modified.

Dosages and dosing schedules are discussed above. It is noted, however, that if the LEVs are administered intravenously, often multiple treatments will be given to the patient, for example, weekly or twice weekly. It will not be unexpected if the therapy continues for about 4 to 16 weeks (4 to 32 treatments) or longer. It is understood that the dosage of LEVs, the frequency of administration, and the length of each treatment course can be adjusted based on clinical or biological responses.

Co-Administration of LEVs with Other Medications

There can be benefits to co-administering LEVs with other medications. For example, use of LEVs to remodel LDL to be less susceptible to aggregation can be favorably combined with a statin, which will lower overall plasma concentrations of LDL.

Exemplary agents that can be combined with LEVs can be selected from the group consisting of an inhibitor of cholesterol synthesis, a statin, simvastatin, atorvastatin, rosuvastatin, a fibrate, an SGLT2 inhibitor, a GLP1 agonist, a DPP4 inhibitor, metformin, a weight-loss drug, a CETP inhibitor, a PCSK9 inhibitor, a cholesterol absorption inhibitor, ezetimibe, low-dose aspirin, an inhibitor of acetyl-CoA carboxylase (ACC), an inhibitor of ATP-citrate lyase (ACL), an LDL-lowering drug, a triglyceride-lowering drug, gemcabene, an inhibitor of sulfatase-2, an inhibitor of sulfatase-2 production or secretion, bempedoic acid, an inhibitor of the microsomal triglyceride transfer protein, an antisense oligonucleotide against APOB mRNA, an inhibitor of the secretion of an apoB-lipoprotein, a fish oil, a fish oil fatty acid, a fish oil fatty acid ester, and a bile-acid binder.

Said other medications can be administered either by their usual route (e.g., statins given by mouth) or they can be incorporated into the LEVs.

### EXAMPLES

### Example 1

### LDL Particles from LEV-Treated Hypercholesterolemic Mice are Far Less Susceptible to SMase-Mediated Aggregation than are LDL Particles from PBS-Treated Hypercholesterolemic Mice

This example is designed to show, in an in-vitro (test-tube) assay, that LDL from LEV-treated mice is far more resistant to SMase-mediated aggregation than is LDL from control (saline-treated) mice. The assay of the susceptibility of LDL to aggregation was performed according to prior literature (9, 10, 16). The example is important because SMase-mediated LDL aggregation is expected to be a major contributor to atherosclerotic plaques associated with cardiovascular disease.

### Production of LEVs

The procedure used to make the LEVs from POPC in this Example was the following: procedures were performed in a sterile biological cabinet, under purified atmosphere (e.g., HEPA-filtered air), with all surfaces and equipment cleaned and sterilized. Synthetic, pure, dry, granular POPC from Avanti Polar Lipids, Inc., was dispersed in sterile, hospital-grade phosphate-buffered saline (However, many different aqueous buffers can be used to manufacture liposomes) by vortexing, to make MLVs, at a concentration of 200 mg POPC per ml.

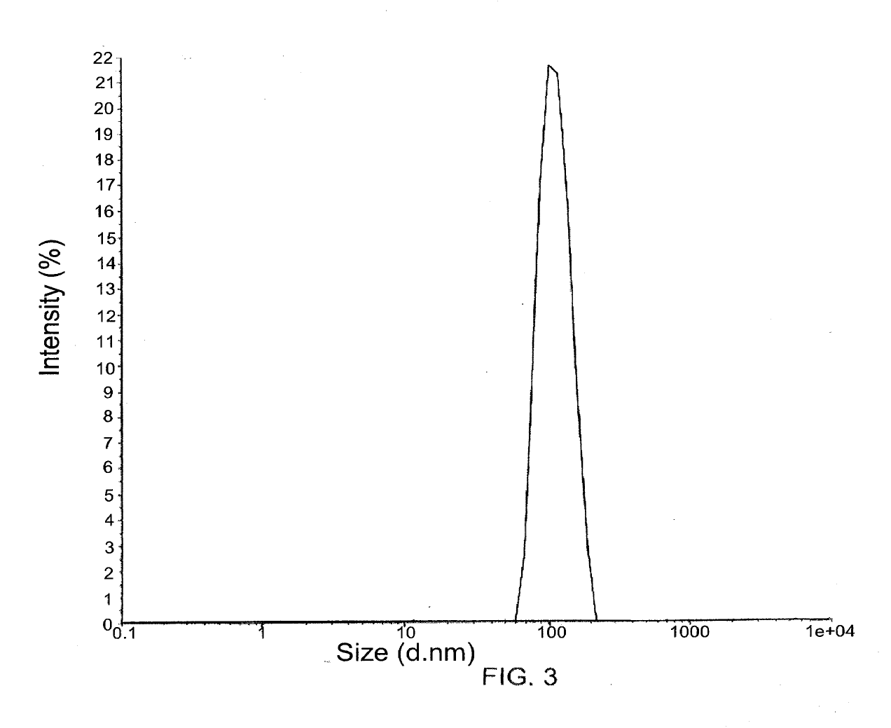

To generate LEVs, the MLVs were extruded 10 times under medium pressures (250 to 300 psi) through two stacked polycarbonate filters (100-nm pore size) that had been fitted into a 10-mL water-jacketed thermobarrel Extruder (Lipex Biomembranes). The LEVs were then filter-sterilized by passage through a 0.45-μm pore-size filter, and an aliquot was verified by endotoxin assay to be endotoxin-free, essentially endotoxin-free, or low-endotoxin (e.g., <0.50 EU/ml). FIG. 3 shows the size distribution by dynamic light scattering (quasi-elastic light scattering) of a typical POPC LEV preparation used in these studies, in which the mean diameter of the LEVs was 116.9 nm, and their size distribution was tightly centered around this mean value.

Treatment of Hypercholesterolemic Mice with Injections of LEVs (or Control Saline Buffer), Isolation of LDL from these Mice, and then Assessment of the Susceptibility of these LDL Samples to Aggregation when Incubated with Sphingomyelinase

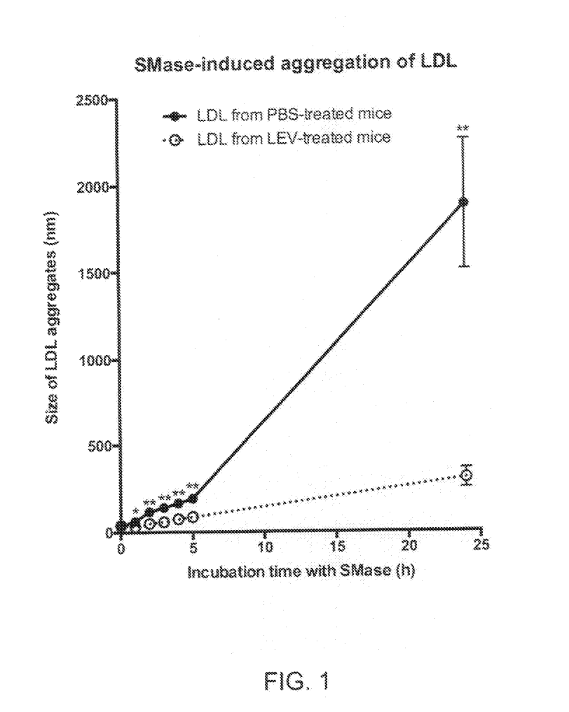

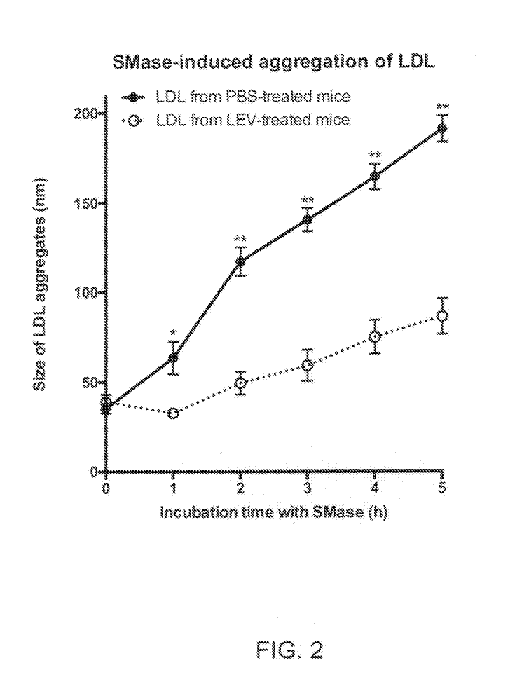

Sixteen hypercholesterolemic human apoB₁₀₀ (huApoB₁₀₀) transgenic mice were randomly divided into two groups of eight mice each. Mice in one group were injected with LEVs at a dose of 1000 mg of POPC per kg of body weight, while mice in the other group were injected with an equivalent volume of PBS (phosphate-buffered saline) solution free of LEVs. The plasma was taken from each mouse one hour later. Each of these 16 plasma samples was raised to a density of 1.063 g/ml and then ultracentrifuged, a process that floats up VLDL, LDL, and, when present, LEVs. The supernatant was subjected to size-exclusion chromatography through a Superose 6 column to separate VLDL and LEVs, which are large, from LDL, which is smaller. To ensure purity of the LDL, some of the LDL samples were passed a second time over the size-exclusion column. The 16 purified LDL samples were each brought to a standard concentration and then incubated with SMase for the indicated times (horizontal axis in FIGS. 1 and 2).

More specifically, the LDL particles (100 μl, 1 mg/ml) were incubated in the wells of microtiter plates at pH 5.5 at 37° C. with human recombinant sphingomyelinase (hrSMase). (For an example of an incubation of LDL with hrSMase, see Sneck M, Nguyen S D, et al. (10).) The mean diameter of the aggregated LDL particles was determined by dynamic light scattering (DLS) at different time points during the incubation (10). The results obtained are summarized in FIGS. 1 and 2.

Aggregation of each LDL sample at each time point was quantified as the average size of the aggregates as determined by dynamic light scattering (vertical axis in FIGS. 1 and 2). Also shown in FIGS. 1 and 2 are SEMs (“standard error of mean”), indicated by error bars (n=8 mice, hence 8 LDL samples, per group). Absence of error bars in FIG. 1 indicates errors smaller than the drawn symbols. Values between the two groups were compared at each time point using Student's two-tailed unpaired t-test. Single asterisks indicate P<0.02 and double asterisks indicate P<0.001, meaning that there was a statistically significant difference at that time point between the mean diameter of LDL-aggregates from LEV-injected mice versus the mean diameter of LDL-aggregates from PBS-injected mice. At t=0, the mean diameters of LDL from the two groups were statistically indistinguishable (P>0.4, i.e., not significant (“ns”)).

### Conclusions

The results of the foregoing experiments demonstrate that LDL from LEV-treated hypercholesterolemic mice are considerably less susceptible to SMase-induced aggregation than is LDL from control (saline-treated) hypercholesterolemic mice. Thus, an injection of LEVs in vivo quickly alters LDL to become less susceptible to aggregation.

### Example 2

### Effect of LEV Treatment on the Composition of LDL

The 16 LDL samples from Example 1 were also subjected to compositional analyses. Lipids were extracted by the Folch procedure, under nitrogen, in the presence of lipid anti-oxidants, and then subjected to an automated, high-throughput tandem mass spectrometry procedure that was previously described in detail (15).

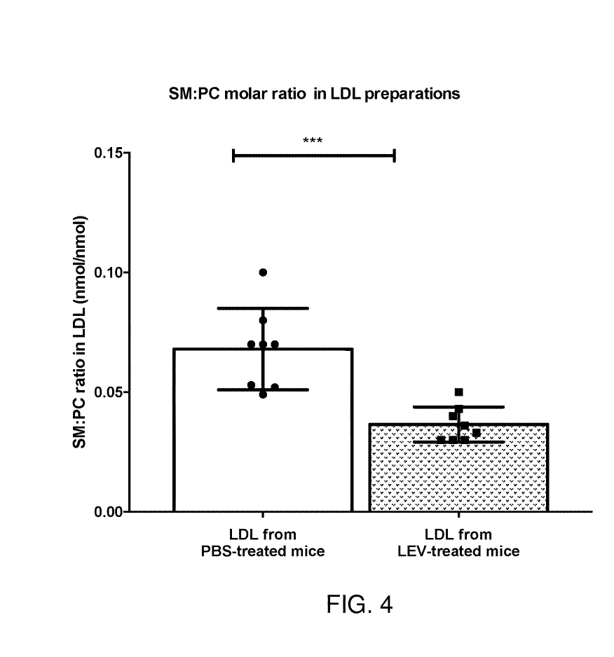

The results are shown in FIG. 4. In FIG. 4, the asterisks indicate that there was a statistically significant difference between the results obtained with mice injected with LEVs and mice injected with PBS. The treatment of mice with a single injection of LEVs resulted in a decrease in the molar ratio of sphingomyelin to phosphatidylcholine (SM:PC) in the LDL of the mice.

There was a statistically significant increase in the overall PC:protein ratio, and there were statistically significant decreases in the UC:PC, UC:protein, and the overall lysoPC:PC ratios in the LDL samples from mice injected with LEVs compared with LDL from mice injected with PBS (control). In addition, the types of PC in the LDL shifted to substantially more POPC.

### REFERENCES

1. Ridker P M, Danielson E, Fonseca F A, Genest J, Gotto A M Jr, Kastelein J J, Koenig W, Libby P, Lorenzatti A J, MacFadyen J G, Nordestgaard B G, Shepherd J, Willerson J T, Glynn R J; JUPITER Study Group. Rosuvastatin to prevent vascular events in men and women with elevated C-reactive protein. N Engl J Med. 2008; 359:2195-207.
2. Cannon C P, Blazing M A, Giugliano R P, McCagg A, White J A, Theroux P, Darius H, Lewis B S, Ophuis T O, Jukema J W, De Ferrari G M, Ruzyllo W, De Lucca P, Im K, Bohula E A, Reist C, Wiviott S D, Tershakovec A M, Musliner T A, Braunwald E, Califf R M; IMPROVE-IT Investigators. Ezetimibe added to statin therapy after acute coronary syndromes. N Engl J Med. 2015; 372:2387-97.
3. Robinson J G, Farnier M, Krempf M, Bergeron J, Luc G, Averna M, Stroes E S, Langslet G, Raal F J, El Shahawy M, Koren M J, Lepor N E, Lorenzato C, Pordy R, Chaudhari U, Kastelein J J; ODYSSEY LONG TERM Investigators. Efficacy and safety of alirocumab in reducing lipids and cardiovascular events. N Engl J Med. 2015; 372:1489-99.
4. Sabatine M S, Giugliano R P, Wiviott S D, Raal F J, Blom D J, Robinson J, Ballantyne C M, Somaratne R, Legg J, Wasserman S M, Scott R, Koren M J, Stein E A; Open-Label Study of Long-Term Evaluation against LDL Cholesterol (OSLER) Investigators. Efficacy and safety of evolocumab in reducing lipids and cardiovascular events. N Engl J Med. 2015; 372:1500-9.
5. Williams, K J, Tabas, I. The response-to-retention hypothesis of early atherogenesis. Arterioscler. Thromb. Vasc. Biol. 1995; 15:551-61.
6. Öörni K, Pentikäinen M O, Ala-Korpela M, Kovanen P T. Aggregation, fusion, and vesicle formation of modified low density lipoprotein particles: molecular mechanisms and effects on matrix interactions. J. Lipid Res. 2000; 41:1703-14.
7. Devlin C M, Leventhal A R, Kuriakose G, Schuchman E H, Williams K J, Tabas I. Acid sphingomyelinase promotes lipoprotein retention within early atheromata and accelerates lesion progression. Arterioscler. Thromb. Vasc. Biol. 2008; 28:1723-30.
8. Rodrigueza W V, Mazany K D, Essenburg A D, Pape M E, Rea T J, Bisgaier C L, Williams K J. (1997). Large versus small unilamellar vesicles mediate reverse cholesterol transport in vivo into two distinct hepatic metabolic pools. Implications for the treatment of atherosclerosis. Arterioscler Thromb Vasc Biol. 1997; 17(10):2132-9.
9 Tabas I, Li Y, Brocia R W, Xu S W, Swenson T L, Williams K J. Lipoprotein lipase and sphingomyelinase synergistically enhance the association of atherogenic lipoproteins with smooth muscle cells and extracellular matrix. A possible mechanism for low density lipoprotein and lipoprotein(a) retention and macrophage foam cell formation. J Biol Chem.1993; 268:20419-32.
10. Sneck M, Nguyen S D, Pihlajamaa T, Yohannes G, Riekkola M-L, Milne R, Kovanen P T, Öörni K. Conformational changes of apoB-100 in SMase-modified LDL mediate formation of large aggregates at acidic pH. J Lipid Res. 1012; 53(9):1832-9.
11. Piepoli M F, Hoes A W, Agewall S, Albus C, Brotons C, Catapano A L, Cooney M T, Corrà U, Cosyns B, Deaton C, Graham I, Hall M S, Hobbs F D, Løchen M L, Löllgen H, Marques-Vidal P, Perk J, Prescott E, Redon J, Richter D J, Sattar N, Smulders Y, Tiberi M, van der Worp H B, van Dis I, Verschuren W M; Authors/Task Force Members. 2016 European Guidelines on cardiovascular disease prevention in clinical practice. Eur Heart J. 2016:37:2315-81. doi: 10.1093/eurheartj/ehw106.
12. Allured M. McCutcheon's 2015 Emulsifiers and Detergents: North American Edition. Princeton, Wis.: MC Publishing Co; 2015.
13. Bloomfield V A. Quasi-elastic light scattering applications in biochemistry and biology. Annu Rev Biophys Bioeng. 1981; 10:421-50.
14. Hope M J, Nayar R, Mayer L D and Cullis P R. Reduction of liposome size and preparation of unilamellar vesicles by extrusion techniques. In: Gregoriadis G, ed. Liposome Technology. 3^{rd} ed. Volume I: Liposome Preparation and Related Techniques. Boca Raton, Fla.: CRC Press; 1993:123-39.
15. Stahlman M, Pham H T, Adiels M, Mitchell T W, Blanksby S J, Fagerberg B, Ekroos K, Borén J. Clinical dyslipidaemia is associated with changes in the lipid composition and inflammatory properties of apolipoprotein-B-containing lipoproteins from women with type 2 diabetes. Diabetologia. 2012; 55:1156-166.
16. Ruuth M, Nguyen S D, Vihervaara T, Laajala T D, Savolainen M, Uusitupa M, Schwab U, Sinisalo J, Aittokallio T, Käkelä R, Jauhiainen M, Kovanen P T and Öörni K. Unstable LDL—Novel mechanism of atherogenesis and link to cardiovascular deaths. Oral presentation at the 22nd Annual Scandinavian Atherosclerosis Conference, Scandinavian Society for Atherosclerosis Research (SSAR), Krogerup Høskole, Humlebaek, Denmark; 15 Apr. 2016.
17. Duivenvoorden R, Tang J, Cormode D P, Mieszawska A J, Izquierdo-Garcia D, Ozcan C, Otten M J, Zaidi N, Lobatto M E, van Rijs S M, Priem B, Kuan E L, Martel C, Hewing B, Sager H, Nahrendorf M, Randolph G J, Stroes E S, Fuster V, Fisher E A, Fayad Z A and Mulder W J. A statin-loaded reconstituted high-density lipoprotein nanoparticle inhibits atherosclerotic plaque inflammation. Nat Commun. 2014; 5:3065. doi: 10.1038/ncomms4065.
18. Gonçalves I, den Ruijter H, Nahrendorf M and Pasterkamp G. Detecting the vulnerable plaque in patients. J Intern Med. 2015; 278:520-30. doi: 10.1111/joim.12414.
19. Borén J and Williams K J. The central role of arterial retention of cholesterol-rich apoB-containing lipoproteins in the pathogenesis of atherosclerosis: a triumph of simplicity Curr Opin Lipidol. 2016, in press. doi: 10.1097/MOL.0000000000000330.
20. Cormode D P, Frias J C, Ma Y, Chen W, Skajaa T, Briley-Saebo K, Barazza A, Williams K J, Mulder W J, Fayad Z A and Fisher E A. HDL as a contrast agent for medical imaging. Clin Lipidol. 2009; 4:493-500. doi: 10.2217/clp.09.38.
21. Guarino A J, Tulenko T N and Wrenn S P. Cholesterol crystal nucleation from enzymatically modified low-density lipoproteins: combined effect of sphingomyelinase and cholesterol esterase. Biochemistry. 2004; 43:1685-93. doi: 10.1021/bi035747r.
22. Duewell P, Kono H, Rayner K J, Sirois C M, Vladimer G, Bauernfeind F G, Abela G S, Franchi L, Nuñez G, Schnurr M, Espevik T, Lien E, Fitzgerald K A, Rock K L, Moore K J, Wright S D, Hornung V and Latz E. NLRP3 inflammasomes are required for atherogenesis and activated by cholesterol crystals. Nature. 2010; 464:1357-61. doi: 10.1038/nature08938.
23. Rajamaki K, Lappalainen J, Öörni K, Välimaki E, Matikainen S, Kovanen P T and Eklund K K. Cholesterol crystals activate the NLRP3 inflammasome in human macrophages: a novel link between cholesterol metabolism and inflammation. PLoS One. 2010; 5:e11765. doi: 10.1371/journal.pone.0011765.
24. Ridker P M. From C-reactive protein to interleukin-6 to interleukin-1: moving upstream to identify novel targets for atheroprotection. Circ Res. 2016; 118:145-56. doi: 10.1161/CIRCRESAHA.115.306656.
25. Ridker P M, Everett B M, Thuren T, MacFadyen J G, Chang W H, Ballantyne C, Fonseca F, Nicolau J, Koenig W, Anker S D, Kastelein J J P, Cornel J H, Pais P, Pella D, Genest J, Cifkova R, Lorenzatti A, Forster T, Kobalava Z, Vida-Simiti L, Flather M, Shimokawa H, Ogawa H, Dellborg M, Rossi P R F, Troquay R P T, Libby P, and Glynn R J, for the CANTOS Trial Group. Antiinflammatory therapy with canakinumab for atherosclerotic disease. N Engl J Med. 2017 (ePub 27 August). doi: 10.1056/NEJMoa1707914.
26. Haka A S, Grosheva I, Chiang E, Buxbaum A R, Baird B A, Pierini L M and Maxfield F R. Macrophages create an acidic extracellular hydrolytic compartment to digest aggregated lipoproteins. Mol Biol Cell. 2009; 20:4932-40. doi: 10.1091/mbc.E09-07-0559.
27. Singh R K, Barbosa-Lorenzi V C, Lund F W, Grosheva I, Maxfield F R and Haka A S. Degradation of aggregated LDL occurs in complex extracellular sub-compartments of the lysosomal synapse. J Cell Sci. 2016; 129:1072-82. doi: 10.1242/jcs.181743.
28. Grosheva I, Haka A S, Qin C, Pierini L M and Maxfield F R. Aggregated LDL in contact with macrophages induces local increases in free cholesterol levels that regulate local actin polymerization. Arterioscler Thromb Vasc Biol. 2009; 29:1615-21. doi: 10.1161/ATVBAHA.109.191882.
29. Sun Y, Ishibashi M, Seimon T, Lee M, Sharma S M, Fitzgerald K A, Samokhin A O, Wang Y, Sayers S, Aikawa M, Jerome W G, Ostrowski M C, Bromme D, Libby P, Tabas I A, Welch C L and Tall A R. Free cholesterol accumulation in macrophage membranes activates Toll-like receptors and p38 mitogen-activated protein kinase and induces cathepsin K. Circ Res. 2009; 104:455-65. doi: 10.1161/CIRCRESAHA.108.182568.
30. Westerterp M, Gautier E L, Ganda A, Molusky M M, Wang W, Fotakis P, Wang N, Randolph G J, D'Agati V D, Yvan-Charvet L and Tall A R. Cholesterol accumulation in dendritic cells links the inflammasome to acquired immunity. Cell Metab. 2017; 25:1294-1304 e6. doi: 10.1016/j.cmet.2017.04.005.
31. Chai S, Chai Q, Danielsen C C, Hjorth P, Nyengaard J R, Ledet T, Yamaguchi Y, Rasmussen L M and Wogensen L. Overexpression of hyaluronan in the tunica media promotes the development of atherosclerosis. Circ Res. 2005; 96:583-591. doi: 10.1161/01. RES.0000158963.37132.8b.
32. Seike M, Ikeda M, Matsumoto M, Hamada R, Takeya M and Kodama H. Hyaluronan forms complexes with low density lipoprotein while also inducing foam cell infiltration in the dermis. J Dermatol Sci. 2006; 41:197-204. doi: 10.1016/j.jdermsci.2005.10.008.
33. Ontong P, Hatada Y, Taniguchi S, Kakizaki I and Itano N. Effect of a cholesterol-rich lipid environment on the enzymatic activity of reconstituted hyaluronan synthase. Biochem Biophys Res Commun. 2014; 443:666-71. doi: 10.1016/j.bbrc.2013.12.028.
34. Hermansson A, Ketelhuth D F J, Strodthoff D, Wurm M, Hansson E M, Nicoletti A, Paulsson-Berne G and Hansson G K. Inhibition of T cell response to native low-density lipoprotein reduces atherosclerosis. J Exp Med. 2010; 207:1081-93. doi: 10.1084/jem.20092243.
35. Sneck M, Nguyen S D, Pihlajamaa T, Yohannes G, Riekkola M-L, Milne R, Kovanen P T and Öörni K. Conformational changes of apoB-100 in SMase-modified LDL mediate formation of large aggregates at acidic pH. J Lipid Res. 2012; 53:1832-9. doi: 10.1194/jlr.M023218.
36. Harrington R A. Targeting inflammation in coronary artery disease. N Engl J Med. 2017 (ePub 27 August). doi: 10.1056/NEJMe1709904.
37. Husten L. Experts caution on CANTOS and canakinumab's future—modest treatment effect in heart disease and infection risk, but anti-cancer signal sparks interest. Medpage Today. 2017 (27 August). https://www.medpagetoday.com/Cardiology/CardioBrief/67534?xid=nl_mpt_cardiodaily_2017-08-28&eun=g491951d0r.

1. A method of decreasing the susceptibility of atherogenic lipoprotein particles to aggregation induced by a sphingomyelinase (SMase) in an animal that comprises a SMase and said atherogenic lipoprotein particles, said method comprising administering vesicles (or liposomes) to said animal so as to cause a decrease in said susceptibility, provided said vesicles or liposomes do not comprise significant amounts of sphingomyelin or unesterified cholesterol, and wherein a human is considered to be an animal, and wherein the animal comprises a closed circulatory system that comprises an artery.

2. A method of claim 1, wherein the method is applied to a human.

3. A method of claim 1 wherein the vesicles are administered parenterally.

4. A method of claim 1 wherein the vesicle is an LEV.

5. A method of claim 1, wherein the vesicles comprise one or more phospholipids, provided the vesicles do not comprise significant amounts of sphingomyelin or unesterified cholesterol.

6-8. (canceled)

9. A method of claim 1 wherein the atherogenic lipoprotein particle is selected from the group consisting of LDL, remnant lipoproteins, cholesterol- and triglyceride-rich remnant lipoproteins (together, referred to C-TRLs), very low-density lipoprotein (VLDL), small VLDL (sVLDL), cholesterol-rich remnant lipoproteins, ß-VLDL, VLDL remnants, chylomicron remnants, postprandial remnants, intermediate-density lipoprotein (IDL), lipoprotein(a) [Lp(a)], and triglyceride-rich lipoproteins (TRL).

10-17. (canceled)

18. A method of claim 1 wherein the susceptibility to aggregation is measured using an assessment system is measured using an assessment system and wherein the assessment system is for assessment of the retention of the atherogenic particles in the arteries in vivo and is selected from the group consisting of an assay of apoB-lipoprotein aggregation and/or retention within the arterial wall in vivo, an imaging method of retained and/or aggregated lipoproteins within the arterial wall, an assay of lipoprotein aggregation and/or retention in a healthy arterial segment, an assay of lipoprotein aggregation and/or retention in a diseased arterial segment, an imaging method (such as cardiac catheterization, intravascular ultrasound (IVUS), an MRI, an MRI with contrast, a CT scan, a scan with contrast, an imaging method with a contrast agent wherein said contrast agent comprises a nanoparticle, and a nuclear medicine study), a method that involves injection of said apoB-lipoprotein into an animal, a method that involves labeling of said apoB-lipoprotein followed by its injection into an animal (said animal comprising a human and a non-human animal), and a method that involves assessments of endogenous apoB-lipoproteins in vivo.

19. (canceled)

20. A method claim 1 wherein said method effects at least one change in the composition of the LDL (or other apoB-lipoprotein) of the human or other animal, said change selected from the group consisting of a decrease in the molar ratio of sphingomyelin to phosphatidylcholine (SM:PC), an increase in the molar fraction of PC that is POPC, a decrease in the ratio of unesterified cholesterol to phosphatidylcholine (UC:PC), a decrease in the lysoPC:PC ratio, an increase in the ratio of PC:protein, an increase in the ratio of POPC:protein, an increase in the ratio of PC to apoB, an increase in the ratio of POPC to apoB, an increase in the ratio of PC to cholesteryl ester (PC:ChE), an increase in the ratio of POPC:ChE, an increase in the ratio of PC to triglycerides (PC:TG), an increase in the ratio of POPC:TG, a decrease in the UC:protein ratio, and any other measures that indicate enrichment of LDL (or other apoB-lipoproteins) in PC and/or POPC, and/or depletion in SM, lysoPC, UC, and apoC-III.

21. A method of claim 20 wherein the vesicle or liposome used in the method comprises a phospholipid that is the same as the phospholipid whose molar fraction will be increased in the LDL or other apoB-lipoprotein.

22. A method of claim 1 wherein the method is applied to a human at (moderate, high, or very high) atherosclerotic cardiovascular risk.

23-30. (canceled)

31. A method claim 1 wherein the vesicles or liposomes are administered with at least one other medication.

32. (canceled)

33. A method claim 1 where the human or other animal does not have dyslipidemia.

34. A kit is for decreasing the susceptibility of atherogenic lipoprotein particles to aggregation in a human (or other animal), said kit comprising:
(1) vesicles; and
(2) printed notice that the kit can be used to decrease the susceptibility of atherogenic lipoprotein particles to aggregation in a human (or other animal),
wherein the vesicles do not comprise sphingomyelin, and wherein the printed notice may be on sheet of paper, a label, or a package or may be obtainable from an internet source (preferably one specified in printed form as part of the kit) such that the printed notice requirement of the kit of the invention is satisfied if the kit comprises a printed notice of where the user can go (for example to a website) to find out that the kit can be used to decrease the susceptibility of atherogenic lipoprotein particles to aggregation in a human (or other animal).

35-36. (canceled)

37. A kit of claim 34 wherein the vesicle is an LEV and the LEV comprises one or more phospholipids, provided the LEV does not comprise significant amounts of sphingomyelin and/or unesterified cholesterol and wherein the kit is intended to reduce the SMase-induced aggregation of atherogenic lipoprotein selected from the group consisting of LDL, remnant lipoproteins, cholesterol- and triglvceride-rich remnant lipoproteins (together, referred to C-TRLs), very low-density lipoprotein (VLDL), small VLDL (sVLDL), cholesterol-rich remnant lipoproteins, ß-VLDL, VLDL remnants, chylomicron remnants, postprandial remnants, intermediate-density lipoprotein (IDL), lipoprotein(a) [Lp(a)], and triglyceride-rich remnant lipoproteins (TRL), and the printed notice optionally may indicate which of those lipoprotein particles the kit is directed at.

38-40. (canceled)

40. A kit of claim 34 wherein the kit is intended to reduce the SMase-induced aggregation of atherogenic lipoprotein: LDL, remnant lipoproteins, cholesterol- and triglyceride-rich remnant lipoproteins (together, referred to C-TRLs), very low-density lipoprotein (VLDL), small VLDL (sVLDL), cholesterol-rich remnant lipoproteins, ß-VLDL, VLDL remnants, chylomicron remnants, postprandial remnants, intermediate-density lipoprotein (IDL), lipoprotein(a) [Lp(a)], and triglyceride-rich remnant lipoproteins (TRL), and the printed notice optionally may indicate which of those lipoprotein particles the kit is directed at.

41. A kit of claim 34, wherein said kit combined with an assessment system, said system being an assessment system for measuring the susceptibility to aggregation of the atherogenic lipoprotein particles and/or their retention by arteries.

42-43. (canceled)

44. A method of claim 1, wherein said method is for decreasing a proatherogenic parameter in an animal, said parameter selected from the group consisting of the formation of cholesterol crystals, abnormal cholesterol-enrichment of cell membranes, and denaturation of apoB, said method comprising administering the vesicles (or liposomes) to said animal so that the amount of said parameter formed, enriched or denatured over a given time period is less than it would be in the absence of said administration, provided said vesicles or liposomes do not comprise significant amounts of sphingomyelin or unesterified cholesterol, and wherein a human is considered to be an animal, and wherein the animal comprises a closed circulatory system that comprises an artery.

45. A method of claim 1, wherein said method is for of decreasing the amount of a proatherogenic parameter, in an animal, said method comprising administering the vesicles (or liposomes) to said animal, so that the amount of said proatherogenic parameter formed over a given time period is less than it would be in the absence of said administration, provided said vesicles or liposomes do not comprise significant amounts of sphingomyelin or unesterified cholesterol, and wherein a human is considered to be an animal, and wherein the animal comprises a closed circulatory system that comprises an artery, wherein said proatherogenic parameter is selected from the group consisting of inflammasome activation, activation of the NLRP3 inflammasome, activation of proatherogenic T-cells, release of harmful cytokines (such as interleukin (IL)-1 beta (IL1β) and IL6), plaque progression, plaque destabilization, and release of C-reactive protein (CRP).

46. A kit is for decreasing the susceptibility of atherogenic lipoprotein particles to aggregation in a human (or other animal), said kit comprising:
(1) vesicles; and
(2) printed notice that the kit can be used to decrease a proatherogenic parameter in human or other animal, said parameter selected from the group consisting of the formation of cholesterol crystals, abnormal cholesterol-enrichment of cell membranes, and denaturation of apoB,
wherein the vesicles do not comprise sphingomyelin, and wherein the printed notice may be on sheet of paper, a label, or a package or may be obtainable from an internet source (preferably one specified in printed form as part of the kit) such that the printed notice requirement of the kit of the invention is satisfied if the kit comprises a printed notice of where the user can go (for example to a website) to find out that the kit can be used to decrease a proatherogenic parameter in human or other animal, said parameter selected from the group consisting of the formation of cholesterol crystals, abnormal cholesterol-enrichment of cell membranes, and denaturation of apoB.
